(19) United States
(12) Patent Application Publication
(10) Pub. No.: US 2026/0360549 A1
(43) Pub. Date: Nov. 12, 2026

(54) CAUSATIVE BEHAVIOR ESTIMATION DEVICE, METHOD AND PROGRAM

(71) Applicant: NTT, Inc., Tokyo (JP)

(72) Inventors: Tomu TOMINAGA, Musashino-shi (JP); Shuhei YAMAMOTO, Musashino-shi (JP); Takeshi KURASHIMA, Musashino-shi (JP)

(73) Assignee: NTT, Inc., Tokyo (JP)

(21) Appl. No.: 19/129,688

(22) PCT Filed: Nov. 15, 2022
(86) PCT No.: PCT/JP2022/042372

§ 371 Date: May 14, 2025

(51) Int. Cl. G16H 50/30

(52) U.S. Cl. CPC G16H 50/30

(57) ABSTRACT

According to an embodiment, a causative behavior estimation device includes first, second, third, and fourth processing units. The first processing unit acquires data indicating a health state, lifestyles, and a working form of a target user for each preset given period. The second processing unit extracts a feature from the data and outputs the extracted feature as a first variable. The third processing unit calculates a feature representative value of the working form from the feature for each lifestyle and outputs the calculated feature representative value as a second variable. The fourth processing unit derives a parameter indicating an extent of influence of the lifestyles on a change in the health state of the working form by giving the first variable and the second variable to a model indicating a relation between the health state of the working form and the lifestyles.

### TECHNICAL FIELD

One aspect of the present invention relates to a causative behavior estimation device, method, and program used to estimate behaviors that are factors influencing health states of users, for example, for each working form.

### BACKGROUND ART

Changes in working forms due to new types of coronavirus infection (hereinafter referred to as COVID-19) influence lifestyles and health management behaviors of people. For example, Non Patent Literature (NPL) 1 reports that people who have started telecommuting under the influence of COVID-19 have lower serious levels of sleep disorder than people who have not started telecommuting according to a questionnaire survey for 13,989 Italian people. In NPL 2, 988 response results collected by an on-line investigation system suggest that, people who had started telecommuting under the influence of COVID-19 have an increased amount of exercise, a decreased intake of junk food, and increased conversation time with colleagues, compared before telecommuting starts, which contribute to an improvement in physical well-being and mental well-being.

These analysis results are important know-ledge for understanding the influence of COVID-19 on the lives and health of people.

### CITATION LIST

### Non Patent Literature

Non Patent Literature 1: Salfi, F., Lauriola, M., D'Atri, A. et al. “Demographic, psychological, chronobiological, and work-related predictors of sleep disturbances during the COVID-19 lockdown in Italy.” Sci Rep 11, 11416 (2021), Internet <URL: https://doi.org/10.1038/s41598-021-90993-y>
Non Patent Literature 2: Xiao, Yijing; Becerik-Gerber, Burcin DDes; Lucas, Gale PhD; Roll, Shawn C. PhD. “Impacts of Working From Home During COVID-19 Pandemic on Physical and Mental Well-Being of Office Workstation Users.” Journal of Occupational and Environmental Medicine: March 2021, Volume 63—Issue 3—p 181-190 doi: 10.1097/JOM.0000000000002097.

### SUMMARY OF INVENTION

### Technical Problem

Incidentally, in NPLs 1 and 2, working forms and living forms are merely compared between a period before the prevalence of COVIT-19 and a period after prevalence of COVIT-19, and an influence of the working forms of home or office on a relation between daily lifestyles and health states cannot be known from the analysis results. That is, users cannot know how differences in the daily working forms of the users causes changes in lifestyles, that is, behaviors, and how the changes in the behaviors influence health states.

In view of the foregoing circumstances, the present invention provides a technique capable of estimating a behavior that is a factor influencing a change in a health state of a user for each working form.

### Solution to Problem

In order to solve the above problem, according to an aspect of the present invention, in a causative behavior estimation device or a causative behavior estimation method, data indicating a health state, a plurality of lifestyles, and a working form of a target user is acquired for each preset given period; a feature is extracted from each piece of acquired data and the feature is set as a first variable; and a feature representative value of each working form is calculated from the feature for each of the plurality of lifestyles and the feature representative value is set as a second variable. Then, a parameter indicating an extent of influence of the plurality of lifestyles on a change in the health state of each working form is derived by giving the first and second variables to a model indicating a relation between the health state of each working form and the plurality of lifestyles; and information indicating a behavior that is a factor of the change in the health state of each working form is generated and output based on the derived parameter.

According to the aspect of the present invention, importance of each lifestyle influencing a health change is estimated for each working form, and an estimation result is suggested to a user. Therefore, the user can specify a lifestyle in which the user takes care of her or his health, that is, a behavior that is a factor of the health change, for each working form of the user. Further, data regarding the health state, the lifestyle, and the working form is acquired for each given period, and the estimation result of the importance of each lifestyle is updated based on the data at each time. Therefore, the lifestyle in which the user is careful can be dynamically suggested again to the user according to a health change for each given period.

### Advantageous Effects of Invention

That is, according to an aspect of the present invention, it is possible to provide a technique through which a behavior that is a factor influencing a change in a health state of a user can be estimated for each working form.

### BRIEF DESCRIPTION OF DRAWINGS

FIG. 1 is a diagram illustrating an example of a system including a causative behavior estimation device according to an embodiment of the present invention.

FIG. 2 is a block diagram illustrating an example of a hardware configuration of the causative behavior estimation device according to the embodiment of the present invention.

FIG. 3 is a block diagram illustrating an example of a software configuration of the causative behavior estimation device according to the embodiment of the present invention.

FIG. 4 is a flowchart illustrating an example of a processing procedure and processing content of causative behavior estimation processing performed by a control unit of the causative behavior estimation device illustrated in FIG. 3.

FIG. 5 is a flowchart illustrating an example of a processing procedure and processing content of model training processing in the processing procedure illustrated in FIG. 4.

FIG. 6 is a flowchart illustrating an example of a processing procedure and processing content of processing for generating and outputting information indicating an estimation result of a causative behavior in the processing procedure illustrated in FIG. 4.

FIG. 7 is a diagram illustrating an example of information indicating an estimation result of a causative behavior generated in the processing illustrated in FIG. 6.

### DESCRIPTION OF EMBODIMENTS

Hereinafter, embodiments of the present invention will be described with reference to the drawings.

### Embodiment

### Configuration Example

### (1) System

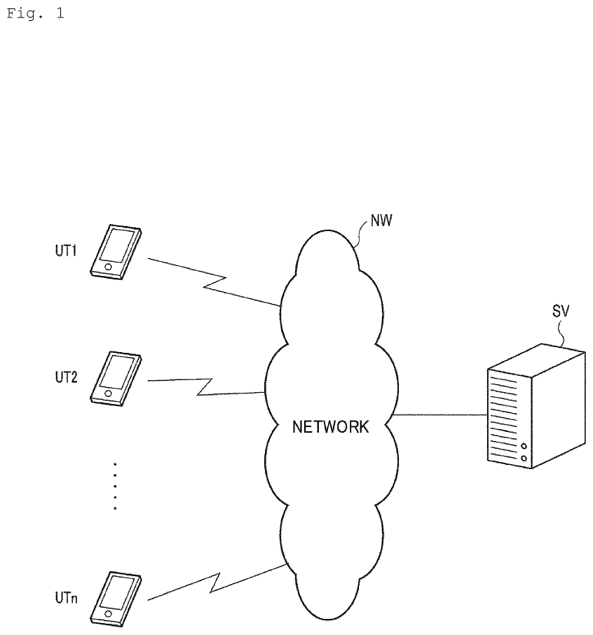

FIG. 1 is a diagram illustrating an example of a configuration of a causative behavior estimation system according to an embodiment of the present invention.

The causative behavior estimation system according to the embodiment of the present invention includes a causative behavior estimation device SV and is capable of transmitting information data via a network NW between the causative behavior estimation device SV and user terminals UT1 to UTn used by a plurality of users using the system.

The user terminals UT1 to UTn are configured with, for example, smartphones, doublet type terminals, or personal computers and include communication tools such as browsers or mailers.

The user terminals UT1 to UTn have functions of acquiring data indicating health states of the users, data indicating a lifestyle, and data indicating a working form from sensors, and transmitting the acquired data to the causative behavior estimation device SV via the network NW as functions according to the embodiment of the present invention. The user terminals UT1 to UTn have a function of receiving information indicating a causative behavior estimation result transmitted from the causative behavior estimation device SV via the network NW.

The data indicating the health state includes, for example, a body weight, a blood pressure, or a blood sugar level, and is measured by, for example, a vital sensor built in any of the user terminals UT1 to UTn or a vital sensor provided in a wearable terminal worn by a user.

The data indicating the lifestyle includes, for example, data indicating the number of steps, a sleep time, a meal volume and a mood, for example, and is measured by an application program provided in any of the user terminals UT1 to UTn.

The data indicating a working form includes, for example, the presence or absence of working for each day, a working start time, a working end time, and a working place. The data is input by, for example, a user and is managed by schedulers provided in the user terminals UT1 to UTn.

The network NW includes, for example, a wide area network of which a core is the Internet and an access network for access to the wide area network. As the access network, for example, a public communication network using wired or wireless connection, a local area network (LAN) using wired or wireless connection, or a cable television (CATV) network is used. When the system is operated, for example, in a company or a business office, the network NW is configured by a private network such as a LAN or a wireless LAN.

### (2) Causative Behavior Estimation Device SV

The causative behavior estimation device SV is configured by, for example, a server computer arranged on a Web or a cloud.

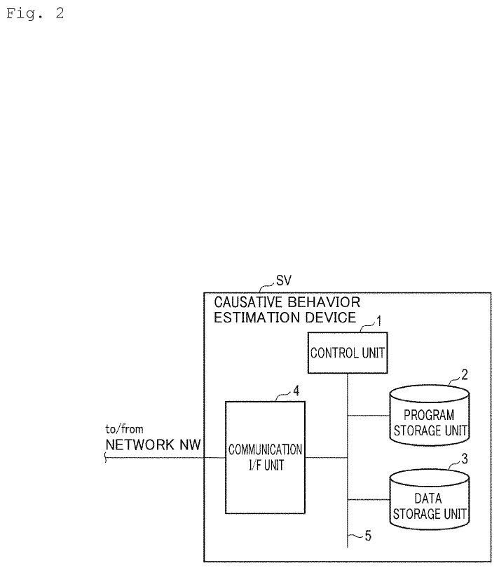

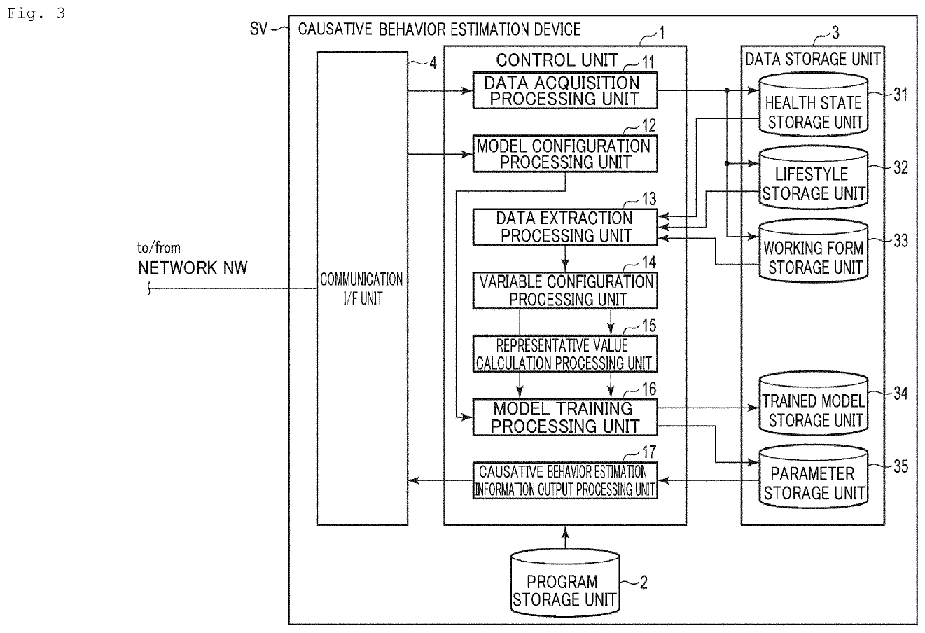

FIGS. 2 and 3 are block diagrams illustrating examples of a hardware configuration and a software configuration of the causative behavior estimation device SV, respectively.

The causative behavior estimation device SV includes a control unit 1 in which a hardware processor such as a central processing unit (CPU) is used. A storage unit including a program storage unit 2 and a data storage unit 3 and a communication interface (hereinafter abbreviated as an “I/F”) unit 4 are connected to the control unit 1 via a bus 5.

The communication I/F unit 4 transmits and receives information data to and from the user terminals UT1 to UTn in conformity with a communication protocol defined by the network NW under the control of the control unit 1.

The program storage unit 2 is configured as a storage medium, for example, by combining a nonvolatile memory such as a solid state drive (SSD) capable of frequent writing and reading and a nonvolatile memory such as a read only memory (ROM), and stores an application program necessary for executing various types of control processing according to the embodiment in addition to middleware such as an operating system (OS). Hereinafter, the OS and each application program will be collectively referred to as a program.

The data storage unit 3 is configured as a storage medium, for example, by combining a nonvolatile memory such as an SSD capable of frequent writing and reading and a volatile memory such as a random access memory (RAM), and includes a health state storage unit 31, a lifestyle storage unit 32, a working form storage unit 33, a trained model storage unit 34, and a parameter storage unit 35 in a storage area as main storage units necessary for embodying an embodiment of the present invention.

The health state storage unit 31, the lifestyle storage unit 32, and the working form storage unit 33 store data indicating a health state, a lifestyle, and a working form of each user acquired from the user terminals UT1 to UTn in association with identification information (hereinafter referred to as user IDs) of users or the user terminals UT1 to UTn.

The trained model storage unit 34 stores learned data that forms a trained model.

The parameter storage unit 35 stores the learned parameter generated through the model training processing by the control unit 1 as an estimation result of the extent of influence of the lifestyle on the health state.

The control unit 1 includes a data acquisition processing unit 11, a model configuration processing unit 12, a data extraction processing unit 13, a variable configuration processing unit 14, a representative value calculation processing unit 15, a model training processing unit 16, and a causative behavior estimation information output processing unit 17 as processing functions necessary to carry out the embodiment of the present invention.

Each of the processing units 11 to 17 of the control unit 1 is implemented by causing a hardware processor of the control unit 1 to execute an application program stored in the program storage unit 2. Some or all of the processing units 11 to 17 may also be implemented using hardware such as a large scale integration (LSI) or an application specific integrated circuit (ASIC).

The data acquisition processing unit 11 receives each piece of data indicating a health state, a lifestyle, and a working form transmitted on each day, for example, from the user terminals UT1 to UTn via the communication I/F unit 4, and stores each piece of received data indicating the health state, the lifestyle, and the working form in the health state storage unit 31, the lifestyle storage unit 32, and the working form storage unit 33 in association with a user ID of a transmission source.

The model configuration processing unit 12 receives data indicating a model structure of the trained model from a manager terminal (not illustrated) used by, for example, a system manager via the communication I/F unit 4. Then, the received data indicating the model structure is given to the model training processing unit 16.

The data extraction processing unit 13 reads each piece of data indicating a health state, a lifestyle, and a working form for each user from the health state storage unit 31, the lifestyle storage unit 32 and the working form storage unit 33. Then, a set of temporally corresponding data among the read data is extracted.

The variable configuration processing unit 14 calculates a feature from each piece of data indicating the temporally corresponding health state, lifestyle, and working form extracted by the data extraction processing unit 13. At this time, when a plurality of types of lifestyles are acquired, the variable configuration processing unit 14 calculates a feature for each of the lifestyles. Then, the variable configuration processing unit 14 transfers each calculated feature related to the health state, the lifestyle, and the working form to the model training processing unit 16.

The representative value calculation processing unit 15 receives a feature related to each lifestyle among the features related to the health state, each lifestyle, and the working form calculated by the variable configuration processing unit 14. Then, a representative value of each working form is calculated for each of the received features related to each lifestyle.

The model training processing unit 16 performs training processing by inputting each feature related to the health state, the lifestyle, and the working form calculated by the variable configuration processing unit 14 and a representative value of the feature of each lifestyle for each working form calculated by the representative value calculation processing unit 15 to the model structure given from the model configuration processing unit 12, and calculates a parameter corresponding to each lifestyle influencing a change in the health state. An example of model training processing will be described in an operation example.

Then, the model training processing unit 16 stores the calculated parameter corresponding to each lifestyle along with the data indicating the model structure when the parameter is obtained, each feature related to the health state, the lifestyle, and the working form and the representative value of the feature for each lifestyle in the trained model storage unit 34. The model training processing unit 16 stores, in addition to these, the parameter corresponding to each lifestyle as information indicating an estimation result of an extent of influence of the lifestyle regarding the health state in the parameter storage unit 35.

The causative behavior estimation information output processing unit 17 reads a parameter corresponding to each lifestyle from the parameter storage unit 35, generates display data indicating the importance of each lifestyle to the change in the health state based on the read parameter corresponding to each lifestyle, and transmits the generated display data from the communication I/F unit 4 to the corresponding user terminals UT1 to UTn. An example of the causative behavior estimation processing will be described in an operation example.

### Operation Example

Next, an operation example of the causative behavior estimation device SV configured as described above will be described.

### (1) Acquisition of Each Piece of Data Regarding User

In the user terminals UT1 to UTn, for example, data related to the health state, the lifestyle, and the working form of the user for one day is collected for each day, data indicating the characteristics, for example, a representative value or an average value, is obtained from each piece of the collected data, and the obtained data is transmitted to the causative behavior estimation device SV together with a user ID at a daily determined time.

On the other hand, the control unit 1 of the causative behavior estimation device SV receives data related to the health states, the lifestyles, and the working forms transmitted from the user terminals UT1 to UTn via the communication I/F unit 4 under the control of the data acquisition processing unit 11. Then, the received data related to the health states, the lifestyles, and the working forms is sequentially stored in the health state storage unit 31, the lifestyle storage unit 32, and the working form storage unit 33 for each day association with the user ID.

### (2) Estimation of Causative Behavior

The control unit 1 of the causative behavior estimation device SV performs processing for each user as follows to estimate a behavior that is a factor influencing a change in a health state of the user whenever new data of one day is stored in the health state storage unit 31, the lifestyle storage unit 32, and the working form storage unit 33.

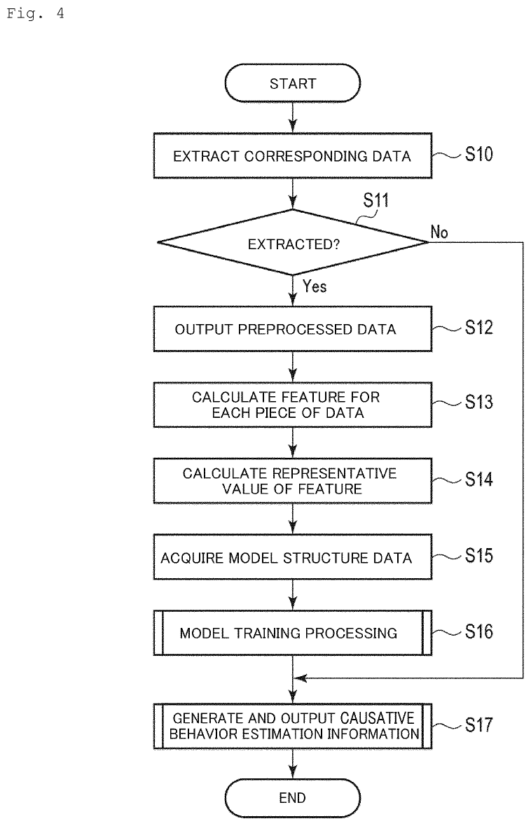

FIG. 4 is a flowchart illustrating an example of a processing procedure and processing content of the causative behavior estimation processing performed by the control unit 1 of the causative behavior estimation device SV.

### (2-1) Data Extraction Processing

The control unit 1 of the causative behavior estimation device SV first reads each piece of data indicating the health state, the lifestyle, and the working form for each user from the health state storage unit 31, the lifestyle storage unit 32 and the working form storage unit 33 under the control of a data extraction processing unit 13 in step S10. Then, the temporally corresponding relation of each piece of read data is arranged, and a set of temporally corresponding data is extracted as an analysis target.

For example, the data extraction processing unit 13 first reads data y_{i,t} indicating a health state, data x_{i,s,k} indicating a lifestyle, and data w_{i,(us,ue)} indicating a working form from the health state storage unit 31, the lifestyle storage unit 32, and the working form storage unit 33, respectively.

Here, an acquisition time of data indicating a health state of a certain user i is defined as t=t₀, t₁, . . . , t_{T} by the following formula.

$$[ Math . 1 ]$$

$${A}_{ij} = \{ {x}_{i , s , k} \vert {t}_{j} < s < {t}_{j - 1} , k ∈ K \} \qquad ( 1 )$$

$${B}_{ij} = \{ {w}_{i ( {u}_{s} {u}_{e} )} \vert {t}_{j} < {u}_{s} , {u}_{e} < {t}_{j + 1} \} \qquad ( 2 )$$

Then, an analytical unit D_{i,j} is configured by the following formula.

$$[ Math . 2 ]$$

$${D}_{ij} = \{ \begin{matrix}\{ {y}_{i , {t}_{j}} , {y}_{i , {t}_{j + 1}} , {A}_{ij} , {B}_{ij} \} , & {A}_{ij} \neq ϕ , {B}_{ij} \neq ϕ \\ ϕ , & otherwise\end{matrix} \qquad ( 3 )$$

Here, (j=0, . . . , T−1).

This indicates a procedure for configuring data as an analysis unit Dᵢⱼ by associating a set A_{i,j} of data indicating a lifestyle acquired between acquisition times tⱼ and tⱼ₊₁ with a set Bᵢⱼ of data indicating a working form when there is the data indicating two health states acquired continuously by a certain user i.

In step S11, the data extraction processing unit 13 determines whether the following set D that has the analysis unit Dig as an element is configured.

[Math. 3]

D={Dᵢⱼ|i=0, . . . ,N:j=0, . . . , T−1} (4)

When the set D of temporally corresponding data can be configured, the data extraction processing unit 13 transfers the configured set D of the data as preprocessed data to the variable configuration processing unit 14 in step S12.

Conversely, when D=φ, that is, when the set D of temporally corresponding data cannot be constituted, the data extraction processing unit 13 moves to output processing of the causative behavior estimation result of step S17.

### (2-2) Configuration of Variable

When the set D of the data is received from the data extraction processing unit 13, the variable configuration processing unit 14 configures the variables for training the trained model in each of the health state, the lifestyle, and the working form 1 in step S13, for example, as follows.

### (2-2-1) Variables Related to Health State

The variable configuration processing unit 14 first calculates a difference between pieces of data y_{i,tj} and y_{i,tj+1} indicating two health states included in the analysis unit Dᵢⱼ by the following formula.

[Math. 4]

y_{i,j}=y_{i,tj+1}y_{i,tj} (5)

The calculated result y_{i,j} is set as a feature of the data indicating the health state. However, when the feature is a scale expressing a difference between y_{i,tj} and y_{i,tj+1} which are the data indicating two health states, another scale other than the scale may be used as a feature.

### (2-2-2) Variable Related to Lifestyle

The variable configuration processing unit 14 calculates a measured value related to a certain lifestyle per unit time acquired at time tⱼ<s<tⱼ₊₁ of a certain user i in the following set corresponding to the analysis unit Dᵢⱼ by the following formula.

A_{i,j}={x_{i,s,k}|tⱼ<s<tⱼ₊₁, k∈K}

### [Math. 5]

$${x}_{i , j , k} = \frac{1}{{t}_{j + 1} - {t}_{j}} {\sum }_{{x}_{i , s , k} ∈ {A}_{ij}} {x}_{i , s , k} \qquad ( 6 )$$

The calculated x_{i,j,k} is set as a feature of a lifestyle k.

However, for example, as long as the feature is a scale indicating a representative value of a measured value of the certain lifestyle k acquired at a time tⱼ<s<tⱼ₊₁ of the certain user i, such as an average value of measured values, a scale other than the above scale may be used as the feature of the data indicating the lifestyle. A different scale may be used for each of a plurality of lifestyles.

### (2-2-3) Variable Related to Working Form

The variable configuration processing unit 14 finally calculates the feature of the data related to the working form as follows.

For example, it is assumed that there are c types of working forms (w={wᵢ, . . . , w_{c}}). In this case, the variable configuration processing unit 14 sets a working form in which a longest time is acquired as a working form of Dᵢⱼ when working hours are totaled for each of the c kinds of working forms in the working form data included in Bᵢⱼ corresponding to the analysis unit Dᵢⱼ.

A total WTc of measurement times of the working forms is calculated as the following formula from the working form data included in Bᵢⱼ.

[Math. 6]

WT_{c}=Σ_{w}_{_{i,(us,uc)}}=w_{c}_{ⁱ}w_{i,(us,uc)}∈Bᵢⱼuₛ−uₛ (7)

Here, c=1, . . . , C.

Based on Formula (7), the working form of Dᵢⱼ is configured as in the following formula.

[Math. 7]

w_{i,j}=(w_{i,j,c}|c=1, . . . , C)^{T} (8)

Here, the following formula is defined.

$$[ Math . 8 ]$$

$$\begin{matrix}{w}_{i , j , c} = \{ \begin{matrix}1 , & {WT}_{{c}^{′}} < {WT}_{c} ( {c}^{′} = 1 , … , C ; {c}^{′} \neq c ) \\ 0 , & otherwise\end{matrix} & ( 9 )\end{matrix}$$

Here, wᵢⱼ is defined such that an element w_{i,j,c} of c in which WTc is the longest is 1 and the other elements are a one-hot vector that is 0.

The variable configuration processing unit 14 transfers a feature y_{i,j} related to the calculated health state, a feature x_{i,j,k} related to each lifestyle k, and the feature wᵢⱼ related to the working form to the model training processing unit 16.

### (2-3) Calculation of Representative Value Related to Each Lifestyle

The control unit 1 of the causative behavior estimation device SV subsequently receives the feature x_{i,j,k} calculated for each lifestyle from the variable configuration processing unit 14 under the control of the representative value calculation processing unit 15 in step S14. Then, for each received feature x_{i,j,k} related to each lifestyle, the representative value of each working form is calculated as follows.

That is, since a distribution of the features of the lifestyle data differs depending on each working form, it is necessary to control the difference during model training. Accordingly, the representative value calculation processing unit 15 calculates an average value of the features x_{i,j,k} of the lifestyle data k in the certain working form Wc of the certain user i by the following formula.

$$[ Math . 9 ]$$

$$\overline{{x}_{i , k , c}} = \frac{{\sum }_{j ∈} \{ j \vert {w}_{i , j , c} = 1 \} {x}_{i , j , k}}{\vert \{ j \vert {w}_{i , j , c} = 1 \} \vert} \qquad ( 10 )$$

Here, a method of obtaining the representative value of the features of the lifestyle data k in the working form wc of the certain user i is not limited to the above formula. A different method may be used to calculate each representative value for a plurality of lifestyles.

The representative value calculation processing unit 15 transfers the representative value of the feature of each of the calculated lifestyle data to the model training processing unit 16.

### (2-4) Training by Trained Model

The control unit 1 of the causative behavior estimation device SV subsequently executes processing for learning parameters corresponding to each lifestyle using the trained model under the control of the model training processing unit 16 in step S16 as follows.

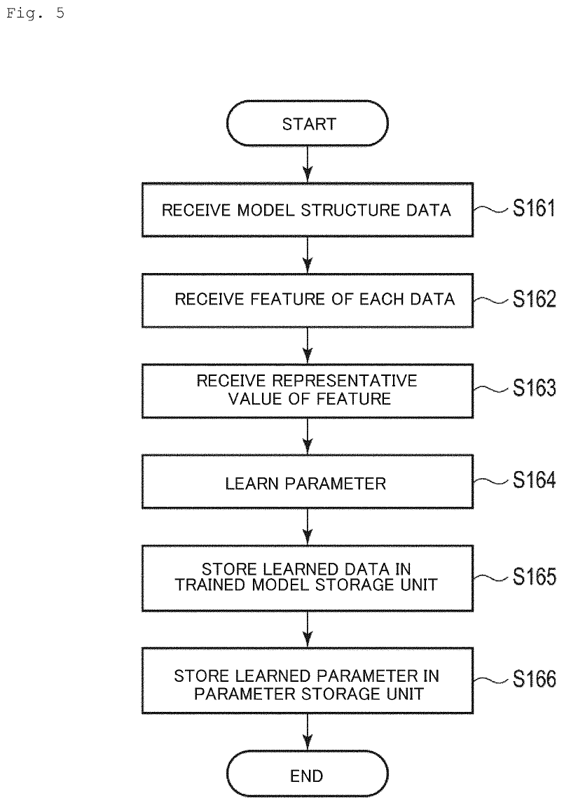

FIG. 5 is a flowchart illustrating an example of a processing procedure and processing content executed by the model training processing unit 16.

### (2-4-1) Configuration of Model Structure

The data indicating the model structure is designated by, for example, a system manager and is transmitted from a manager terminal. On the other hand, the control unit 1 of the causative behavior estimation device SV receives the data indicating the model structure by the model configuration processing unit 12 via the communication I/F unit 4, and transfers the data to the model training processing unit 16.

The model training processing unit 16 first receives the data indicating the model structure from the model configuration processing unit 12 in step S161.

As the model structure, the following model is used to train a relation between the health state and the lifestyle for each working form. In this example, a relation between the feature y_{i,j} related to the health state and the feature x_{i,j,k} related to each lifestyle k is expressed as a regression model indicated by the following formula using a parameter θ.

$$[ Math . 10 ]$$

$${y}_{ij} = {θ}_{o} + {θ}_{1} {x}_{i , j , L} + … + {θ}_{L} {x}_{i , j , L} + {e}_{i , j} = {\sum }_{k ∈ K} {θ}_{k} {x}_{i , j , k} + {e}_{i , j} \qquad ( 11 )$$

$$\begin{matrix}{x}_{t / 0} = 1 & ( 12 )\end{matrix}$$

Here, it is assumed that a parameter θₖ indicates a different value θ_{k,c} according to the working form wc. It is considered that the feature x_{i,j,k} related to the lifestyle in which a working form w_{i,j,c}=1 corresponds to θ_{k,c}.

$$[ Math . 11 ]$$

$${θ}_{k} {x}_{i , j , k} = {\sum }_{c = 1}^{c} {θ}_{k , c} {w}_{i , j , c} {x}_{i , j , k} \qquad ( 13 )$$

Then, the above formula can be expressed.

Further, since the difference in distribution of the feature x_{i,j,k} related to the lifestyle for the working form is controlled, the representative value of the feature x_{i,j,k} related to the lifestyle is used to define the following formula.

[Math. 12]

x_{i,j,k}→x_{i,j,k}−x̅_̅{̅i̅,̅k̅,̅c̅}̅

Accordingly, a regression model indicating a relation between the feature y_{i,j} related to the health state and the feature x_{i,j,k} related to each lifestyle k can be expressed as the following formula.

$$[ Math . 13 ]$$

$${y}_{ij} = {\sum }_{k} \dot{{θ}_{k} {x}_{i , j , k}} + {e}_{i , j} \to {\sum }_{k} {\sum }_{c} \dot{{θ}_{k , c}} {w}_{i , j , c} ( {x}_{i , j , k} - \overline{{x}_{i , k , c}} ) + {e}_{i , j} \qquad ( 14 )$$

In this example, the case in which a linear regression model is generally adopted as a mathematical model with highest interpretation has been described. Any other mathematical model can be adopted as long as the mathematical model is a machine learning model can express an importance that is a feature of each lifestyle as a feature of each lifestyle for the user for each working form.

### (2-4-2) Training of Parameter

Next, the model training processing unit 16 acquires the feature y_{i,j} related to the health state, the feature x_{i,j,k}related to each lifestyle k, and the feature wᵢⱼ related to the working form from the variable configuration processing unit 14 in step S162, and further acquires the representative value of the feature of each piece of lifestyle data from the representative value calculation processing unit 15 in step S163.

Then, in step S164, the model training processing unit 16 executes training processing by inputting, as a variable, each feature related to the health state, the lifestyle, and the working form acquired from the variable configuration processing unit 14 and the representative value of the feature of each lifestyle for each working form acquired from the representative value calculation processing unit 15 in the model structure acquired from the model configuration processing unit 12, and thus calculates the parameter θ indicating the influence of each lifestyle for a change in the health state as follows.

Here, a case in which training is performed using the least squares method which is a most typical method will be described as an example.

First, a vector a=(a₀, . . . , aₙ) that has a₀, . . . , aₙ as elements is denoted by {aᵢ}i. Then, the model structure expressed in Formula (14) is modified to the following formula.

$$[ Math . 14 ]$$

$${y}_{ij} = {\sum }_{k} {\sum }_{c} {θ}_{k , c} {w}_{i , j , c} ( {x}_{i , j , k} - \overline{{x}_{i , k , c}} ) + {e}_{i , j} = \{ {( {{w}_{i , j , c} ( {x}_{i , j , k} - \overline{{x}_{i , k , c}} )}_{c} )}_{k} \} {\{ {\{ {θ}_{k , c} \}}_{c} \}}_{k}^{T} + {e}_{i , j} \qquad ( 15 )$$

$$[ Math . 15 ]$$

$${X = ( {\{ {\{ {w}_{0 , 0 , c} ( {x}_{0 , 0 , k} - \overline{{x}_{0 , k , c}} ) \}}_{c} \}}_{k}^{T} , … , {\{ {w}_{N , T - 1 , c} ( {x}_{N , T - 1 , k} - \overline{{x}_{N , k , c}} ) \}}_{c} \}}_{k}^{T} ) \qquad ( 16 )$$

$$\begin{matrix}y = {\{ {\{ {y}_{i , j} \}}_{j} \}}_{i}^{τ} & ( 17 )\end{matrix}$$

$$Θ = {\{ {\{ {θ}_{k , c} \}}_{c} \}}_{k}^{T} \qquad ( 18 )$$

$$e = {\{ {\{ {e}_{i , j} \}}_{j} \}}_{i}^{T} \qquad ( 19 )$$

Here, when the above formula is defined, Formula (15) is expressed as y=X^{T}Θ+e.

In the least squares method, a parameter for minimizing an error term e of the above formula is calculated.

Here, assuming that an estimated value of y is y \hat{ }=X^{T} Θ, the error term e is defined as follows.

e=y−y\hat{ }=y−X^{T}Θ (20)

By solving the following optimization problem, a desired parameter can be obtained.

$$[ Math . 16 ]$$

$${argmin}_{Θ} {e}^{T} e = {argmin}_{Θ} {( y - {X}^{T} Θ )}^{T} ( y \to {X}^{T} Θ )$$

(21)

Here, a loss function may be defined as to L(Θ)=(y−X^{T} Θ)^{T} (y−X^{T}Θ), and a point at which a gradient of the loss function with respect to Θ is 0 may be found.

$$[ Math . 17 ]$$

$$\begin{matrix}\frac{\partial L ( Θ )}{\partial Θ} = 0 & ( 22 )\end{matrix}$$

$$⇔ - 2 {X}^{T} y + 2 {X}^{T} X Θ = 0 \qquad ( 23 )$$

$$⇔ Θ = {( {X}^{T} X )}^{- 1} {X}^{T} y \qquad ( 24 )$$

From the above, a more optimum parameter Θ is obtained as the following formula.

$$[ Math . 18 ]$$

$${Θ = {\{ ( {θ}_{k , c} \}}_{c} \}}_{1}^{T} .$$

In step S165, the model training processing unit 16 stores the optimum parameter Θ corresponding to each lifestyle in the trained model storage unit 34 along with the data indicating the model structure when the parameter Θ is acquired, each feature related to the health state, the lifestyle, and the working form, and the representative value of the features for each lifestyle.

In step S166, the model training processing unit 16 stores the optimum parameter Θ corresponding to each of the calculated lifestyles in the parameter storage unit 35 as an estimation result of the influence of each lifestyle for the change in the health state of each working form.

### (3) Output of Estimation Result

In step S17, the control unit 1 of the causative behavior estimation device SV finally generates display data indicating the importance of each lifestyle by each working form under the control of the causative behavior estimation information output processing unit 17, and transmits the generated display data to the user terminals UT1 to UTn.

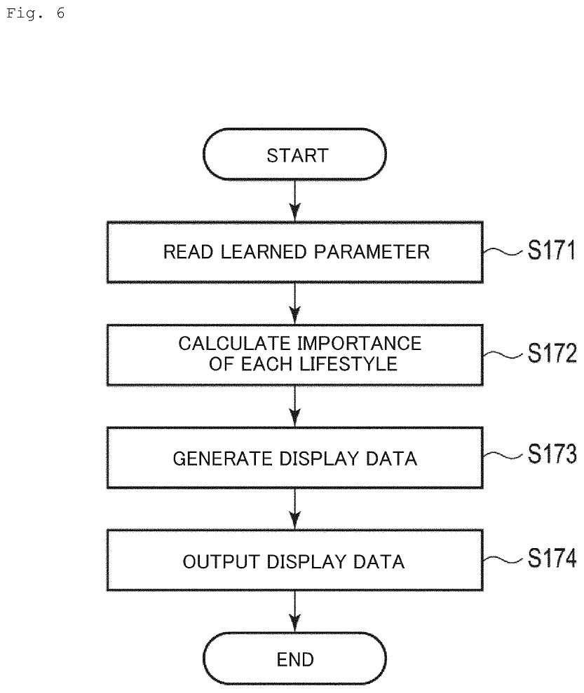

FIG. 6 is a flowchart illustrating an example of the processing procedure and the processing content of the causative behavior estimation information output processing unit 17.

That is, in step S171, the causative behavior estimation information output processing unit 17 first reads a learned parameter Θ corresponding to each lifestyle from the parameter storage unit 35. Then, in step S172, the causative behavior estimation information output processing unit 17 calculates the importance of each lifestyle for the health state of each working form based on the learned parameter Θ corresponding to each read lifestyle.

For example, in a certain working form w_{c}, a ratio of an absolute value of a parameter of the certain lifestyle k to a total sum of absolute values of parameters corresponding to each lifestyle is defined as an importance π_{k,c} of the lifestyle. The ratio is calculated by, for example, the following formula.

$$[ Math . 19 ]$$

$${π}_{k , c} = \frac{\vert {θ}_{k , c} \vert}{{\sum }_{k} \vert {θ}_{k , c} \vert}$$

Hereinafter, a specific example will be described. Now, it is assumed that a daily weight change is a health state and exercise, meal, sleep, and mood are given as lifestyles. Two forms of “home” and “office” are assumed as working forms. In this case, as a result of the above calculation by the causative behavior estimation information output processing unit 17, the importance π_{k,c} of each lifestyle by each working form is calculated as, for example, the followings. π(exercise, home)=0.50, π(meal, home)=0.30, π(sleep, home)=0.15, π(mood, home)=0.05 π(exercise, office)=0.15, π(meal, office)=0.30, π(sleep, office)=0.30, π(mood, office)=0.25

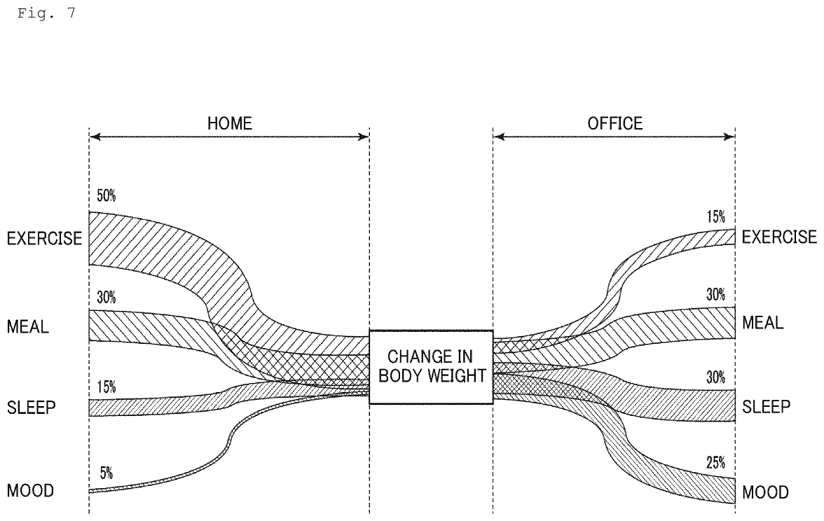

The causative behavior estimation information output processing unit 17 generates display data in step S173 in order to suggest the calculation result of the importance π_{k,c} of each lifestyle by each working form to the user. FIG. 7 illustrates an example of display data generated to suggest a calculation result of the importance π_{k,c} of each lifestyle by each working form to the user.

In step S174, the causative behavior estimation information output processing unit 17 finally transmits the generated display data from the communication I/F unit 4 to the user terminals UT1 to UTn of a target user via the network NW. Based on the display data displayed on the user terminals UT1 to UTn, the target user can specify whether a lifestyle influencing an own weight change, that is, a behavior that is a factor of a health change, is one of the above four types of lifestyles according to the importance.

When a purpose of suggesting an important lifestyle influencing a change in the health state by each working form to the user can be achieved, another method may be used, for example, as long as the importance π_{k,c} is indicated by a bar graph.

In the above example, the case in which the causative behavior estimation information output processing unit 17 transmits the calculation result of the importance π_{k,c} of each lifestyle by each working form to the user terminals UT1 to UTn as display data has been described as an example. However, the present invention is not limited thereto, and the causative behavior estimation information output processing unit 17 may compare, for example, the calculated importance π_{k,c} of each lifestyle with a preset threshold, specify a lifestyle in which the importance π_{k,c} is equal to or more than the threshold, generate display data indicating the specified lifestyle, and transmit the display data to the user terminals UT1 to UTn. At this time, the causative behavior estimation information output processing unit 17 may add the importance π_{k,c} to the specified lifestyle.

### Operations and Effects

As described above, according to an embodiment, the data acquisition processing unit 11 first acquires data indicating the health state and a plurality of lifestyles of the user for each of the working form (for example, home and office), and the data extraction processing unit 13 extracts a set of temporally corresponding data among the data, the variable configuration processing unit 14 obtains the feature of each piece of data included in the set of the data, and the representative value calculation processing unit 15 calculates the representative value of the feature related to each lifestyle by each working form based on each of the obtained features. The model training processing unit 16 applies the feature of each piece of data and the representative value to the model structure acquired by the model configuration processing unit 12, and learns a parameter indicating the importance of each lifestyle influencing the change in the health state of the user. The causative behavior estimation information output processing unit 17 generates the display data indicating the importance of each lifestyle by each working form based on the learned parameter Θ corresponding to each lifestyle obtained by the training and transmits the display data to the user terminals UT1 to UTn.

Accordingly, the importance of each lifestyle influencing the health change is estimated by each working form for each given period, and the estimation result is suggested to the user. Therefore, the user can specify a lifestyle in which the user takes care of her or his health in the own working form, that is, a behavior that is a factor of the health change, for each given period.

### Other Embodiments

In the above-described embodiment, the case in which the processing function of the causative behavior estimation device SV is provided in a server computer on a Web or a cloud has been described as an example. For example, the function may be provided in a server computer disposed on a local area network such as a workplace or a community, a personal computer shared by a plurality of users, and the like. The functions of the causative behavior estimation device SV may be distributed to a plurality of server computers and personal computers used by users or the like.

In addition, types of health state, types of lifestyles to be estimated, types of working forms, a method of calculating a feature of each piece of data, a structure of the trained model, a structure of display data to be suggested to a user, and the like can be variously modified and implemented within a range not deviating from the gist of the present invention.

Although the embodiments of the present invention have been described in detail above, the above description is merely an illustrative of the present invention in all respects. It is needless to say that various improvements and modifications can be made without departing from the scope of the invention. That is, when the present invention is embodied, specific configurations may be adopted as appropriate according to the embodiments.

In short, the present invention is not limited to the above embodiments as they are and can be embodied by modifying constituent elements within a range not departing from the gist of the present invention at the implementation stage. Various inventions can be formed by appropriately combining the plurality of constituent elements disclosed in the above embodiments. For example, some constituent elements may be omitted from all the constituent elements described in the embodiments. Further, constituent elements from different embodiments may be combined as appropriate.

### REFERENCE SIGNS LIST

SV Causative behavior estimation device
UT1 to UTn User terminal
NW Network
1 Control unit
2 Program storage unit
3 Data storage unit
4 Communication I/F unit
5 Bus
11 Data acquisition processing unit
12 Model configuration processing unit
13 Data extraction processing unit
14 Variable configuration processing unit
15 Representative value calculation processing unit
16 Model training processing unit
17 Causative behavior estimation information output processing unit
31 Health state storage unit
32 Lifestyle storage unit
33 Working form storage unit
34 Training model storage unit
35 Parameter storage unit

1. A causative behavior estimation device comprising processing circuitry configured to:
acquire data indicating a health state, a plurality of lifestyles, and a working form of a target user for each preset given period;
extract a feature from each piece of data indicating the health state, the plurality of lifestyles, and the working form and output the extracted feature as a first variable;
calculate a feature representative value of each working form from the feature for each of the plurality of lifestyles and output the calculated feature representative value as a second variable;
derive a parameter indicating an extent of influence of the plurality of lifestyles on a change in the health state of each working form by giving the first variable and the second variable to a model indicating a relation between the health state of each working form and the plurality of lifestyles; and
to generate and output information indicating a behavior that is a factor of the change in the health state of each working form based on the parameter.

2. The causative behavior estimation device according to claim 1, wherein the processing circuitry is configured to:
extract a set of the data corresponding in time from the data indicating the health state, the plurality of lifestyles, and the working form, and
extract the feature from each piece of data included in the extracted set of the data and output the extracted feature as the first variable.

3. The causative behavior estimation device according to claim 1, wherein the processing circuitry is configured to:
use a linear regression model as a model indicating a relation between the health state of the working for and the plurality of lifestyles and calculate the parameter using a least squares method.

4. The causative behavior estimation device according to claim 1, wherein the processing circuitry is configured to:
calculate a ratio of an absolute value of the parameter of an individual lifestyle to a sum of absolute values of parameters of the plurality of lifestyles of each working form, as information indicating importance of the individual lifestyle, and
output the calculated information indicating the importance of the individual lifestyle as the information indicating the behavior that is a factor of the change in the health state.

5. The causative behavior estimation device according to claim 1, wherein the processing circuitry is configured to:
calculate a ratio of an absolute value of the parameter of an individual lifestyle to a sum of absolute values of the parameters of the plurality of lifestyles of each working form, as information indicating importance of the individual lifestyle, and output the lifestyle in which the calculated importance is a preset threshold or more as the information indicating the behavior that is a factor of the change in the health state.

6. A causative behavior estimation method, comprising:
acquiring data indicating a health state, a plurality of lifestyles, and a working form of a target user for each preset given period;
extracting a feature from each piece of data indicating the health state, the plurality of lifestyles, and the working form and outputting the extracted feature as a first variable:
calculating a feature representative value of each working form from the feature for each of the plurality of lifestyles and outputting the calculated feature representative value as a second variable;
deriving a parameter indicating an extent of influence of the plurality of lifestyles on a change in the health state of each working form by giving the first variable and the second variable to a model indicating a relation between the health state of each working mode and the plurality of lifestyles; and
generating and outputting information indicating a behavior that is a factor of the change in the health state of each working form based on the parameter.

7. A non-transitory computer-readable storage medium configured with instructions executable by one or more processors to cause the one or more processors to perform operations comprising:
acquiring data indicating a health state, a plurality of lifestyles, and a working form of a target user for each preset given period;
extracting a feature from each piece of data indicating the health state, the plurality of lifestyles, and the working form and output the extracted feature as a first variable;
calculating a feature representative value of each working form from the feature for each of the plurality of lifestyles and output the calculated feature representative value as a second variable;
deriving a parameter indicating an extent of influence of the plurality of lifestyles on a change in the health state of each working form by giving the first variable and the second variables to a model indicating a relation between the health state of each working form and the plurality of lifestyles; and
generating and outputting information indicating a behavior that is a factor of the change in the health state of each working form based on the parameter.

8. The non-transitory computer-readable storage medium according to claim 7, further comprising:
extracting a set of the data corresponding in time from the data indicating the health state, the plurality of lifestyles, and the working form,
wherein the extracting the feature extracts the feature from each piece of data included in the extracted set of the data and outputs the extracted feature as the first variable.

9. The non-transitory computer-readable storage medium according to claim 7, wherein:
the deriving the parameter uses a linear regression model as a model indicating a relation between the health state of the working form and the plurality of lifestyles and calculates the parameter using a least squares method.

10. The non-transitory computer-readable storage medium according to claim 7, wherein:
the generating and outputting the information calculates a ratio of an absolute value of the parameter of the individual lifestyle to a sum of absolute values of the parameters of the plurality of lifestyles of each working form, as information indicating importance of the individual lifestyle, and outputs the calculated information indicating the importance of the individual lifestyle as the information indicating the behavior that is a factor of the change in the health state.

11. The non-transitory computer-readable storage medium according to claim 7, wherein:
the generating and outputting the information calculates a ratio of an absolute value of the parameter of the individual lifestyle to a sum of absolute values of the parameters of the plurality of lifestyles of each working form, as information indicating importance of the individual lifestyle, and outputs the lifestyle in which the calculated importance is a preset threshold or more as the information indicating the behavior that is a factor of the change in the health state.
